(19) United States
(12) Patent Application Publication
(10) Pub. No.: US 2009/0142307 A1
(43) Pub. Date: Jun. 4, 2009

(54) Shape-Based Approach for Scaffoldless Tissue Engineering

(76) Inventors: Kyriacos A. Athanasiou, Houston, TX (US); Jerry Hu, Houston, TX (US); Adam Aufderheide, Houston, TX (US)

Correspondence Address: BAKER BOTTS, LLP, 910 LOUISIANA, HOUSTON, TX, 77002-4995, US

(21) Appl. No.: 12/246,367

(22) Filed: Oct. 6, 2008

Related U.S. Application Data
(63) Continuation-in-part of application No. 11/571,790.
Application No. 11/571,790 is a continuation-in-part of application No. PCT/US2007/066089, filed on Apr. 5, 2007.
Application No. PCT/US2007/066089 is a continuation-in-part of application No. PCT/US2007/066085, filed on Apr. 5, 2007.
Application No. PCT/US2007/066085 is a continuation-in-part of application No. PCT/US2007/066092, filed on Apr. 5, 2007.
(60) Provisional application No. 60/586,862, filed on Jul. 9, 2004.
(60) Provisional application No. 60/789,851, filed on Apr. 5, 2006.
(60) Provisional application No. 60/789,853, filed on Apr. 5, 2006.
(60) Provisional application No. 60/789,855, filed on Apr. 5, 2006.
(60) Provisional application No. 60/789,851, filed on Apr. 5, 2006.
(60) Provisional application No. 60/789,853, filed on Apr. 5, 2006.
(60) Provisional application No. 60/789,855, filed on Apr. 5, 2006.
(60) Provisional application No. 60/789,851, filed on Apr. 5, 2006.
(60) Provisional application No. 60/789,853, filed on Apr. 5, 2006.
(60) Provisional application No. 60/789,855, filed on Apr. 5, 2006.

(51) Int. Cl. A61F 2/02; C12N 5/06; A61K 35/12

(52) U.S. Cl. 424/93.7; 435/397; 424/423

(57) ABSTRACT

Methods for forming tissue engineered constructs without the use of scaffolds and associated methods of use in tissue replacement. One example of a method may comprise providing a shaped hydrogel negative mold; seeding the mold with cells; allowing the cells to self-assemble in the mold to form a tissue engineered construct.

### CROSS-REFERENCE TO RELATED APPLICATIONS

This application is a continuation-in-part of application Ser. No. 11/571,790 filed Jan. 8, 2007, which claims the benefit of International Application No. PCT/US2005/24269 filed Jul. 8, 2005, which claims the benefit of U.S. Provisional Application Ser. No. 60/586,862 filed on Jul. 9, 2004; and also a continuation-in-part of International Application Nos. PCT/US2007/066089, PCT/US2007/066085, and PCT/US2007/066092 all filed Apr. 5, 2007, and all of which claim the benefit of U.S. Provisional Application Nos. 60/789,851, 60/789,853, and 60/789,855 all filed Apr. 5, 2006, all of which are incorporated herein by reference.

### STATEMENT OF GOVERNMENT INTEREST

This disclosure was developed at least in part using funding from the National Institutes of Health, Grant Number R01 AR47839-2. The U.S. government may have certain rights in the invention.

### BACKGROUND

Tissue engineering is an area of intense effort today in the field of biomedical sciences. The development of methods of tissue engineering and replacement is of particular importance in tissues that are unable to heal or repair themselves, such as hyaline articular cartilage, tissues of the knee meniscus, and tissues of the temporomandibular joint. For example, the meniscus is a load bearing, fibrocartilaginous tissue within the knee joint that is responsible for lubrication, stability, and shock absorption. Regions of the meniscus, namely those in the avascular zone, are virtually incapable of healing or repairing themselves adequately in response to trauma or pathology. Loss of mechanical function of the meniscus is associated with development of degeneration and eventual osteoarthritis.

Because the naturally occurring repair mechanisms are insufficient, researchers have proposed various in vitro approaches to the production of cartilaginous tissue. Generally, most cartilaginous tissue regeneration strategies have been scaffold-based. However, there are disadvantages that come with using either natural or synthetic scaffold materials. Many synthetic polymers can induce inflammatory responses or create a local environment unfavorable to the biologic activity of cells. On the other hand, the major problem associated with natural polymer scaffolds is reproducibility. Moreover, these methods typically involve seeding cultured chondrocytes and/or fibrochondrocytes into a biological or synthetic scaffold. The seeded cells may migrate from the scaffold to the bottom of the culture vessel or well, even if the plates are not treated to promote cell adhesion. Cells plated on non-tissue-treated plates may still eventually attach. Within a week of culture, proteins made by the cells or supplied in the medium have usually adsorbed onto the bottom of the wells to promote attachment. This results in a reduction in the size of the construct. Another drawback is that the attached cells tend to flatten and change to a different phenotype. Those cells compete with the remaining cells for nutrients and do not produce the desired extracellular matrix proteins for tissue regeneration.

### DRAWINGS

A more complete understanding of this disclosure may be acquired by referring to the following description taken in combination with the accompanying figures.

FIG. 1 shows the gross appearance (rows 1 and 2) and histological sections (rows 3 and 4) of 6-mm punched disks from constructs cultured at t=4 wks, 8 wks, and 12 wks over the agarose substratum. Each mark on the ruler is 1 mm. These constructs were flat and smooth. Increases in thickness and opacity over the culture period were observed. Safranin-O/fast green staining for GAGs (row 3) and collagen type II immunohistochemistry (row 4) were observed throughout the constructs at each time point. Chondrocytes rested in lacunae throughout the construct.

FIG. 2 shows the gross appearance (rows 1 and 2) and histological sections (rows 3 and 4) of constructs cultured at t=4 wks and 8 wks on TCP. Each mark on the ruler is 1 mm. In contrast to the constructs cultured over agarose, these constructs are contorted with many folds. Increases in thickness and opacity over the culture period were observed. Safranin-O/fast green staining (row 3) and collagen type II immunohistochemistry (row 4) staining were observed. The constructs contained both dense and diffuse regions.

FIG. 3 shows the total ECM per construct in micrograms. Data are shown as mean±standard deviation, and significance is defined as p<0.05. Significant groups are separated by different letters. Constructs cultured over agarose contained significantly more ECM per construct than constructs cultured on TCP at the same time points. A) Total GAG per construct. Significant increases in GAG per construct were observed for both treatments. B) Total collagen per construct. Significant increases in collagen per construct were observed for both treatments. Due to the absence of immunohistochemistry staining for collagen type I, and also due to gel electrophoresis, most of the collagen produced is considered type II.

FIG. 4 shows the correlation of aggregate modulus (HA) values of native articular cartilage and constructs formed over agarose to GAG/dw and to collagen/dw. Every point represents HA plotted against ECM/dw for a specific time point as indicated by arrows. HA shows a strong positive correlation with collagen/dw (R²=1.00) and a strong negative correlation with GAG/dw (R²=0.99). Since the ECM composed mainly of collagen and GAG, the observed increasing collagen to GAG ratio resulted in decreasing GAG/dw over time and a negative correlation of GAG to HA.

FIG. 5 shows the pressure chamber assembly consisting of a 1.2 L stainless-steel vessel (A) connected to a water-driven piston (B) seated on an Instron 8871 (C). Cells were placed in heat-sealed bags and placed in the stainless-steel vessel (A). The vessel was then placed in an adjacent water bath (not shown). The Instron (C) drove the piston (B) to pressurize the fluid within.

FIG. 6 shows the gross morphology of the self-assembled constructs at t=4 wks and t=8 wks. The cells were seeded without a scaffold and without any ECM at t=0 wks. By accumulating ECM produced by the cells, the constructs rapidly reached more than 1 mm thickness after 4 wks of culture.

FIG. 7 shows the Safranin O staining for GAG (top) and immunohistochemistry staining (bottom) for collagen type II of pressurized constructs and of controls. Both stains were observed throughout the constructs from both treatments. The constructs appeared denser at t=8 wks than t=4 wks for both treatments. By t=8 wks, most of the cells were found to reside in lacunae (arrows).

FIG. 8 shows the total GAG per construct over the 8-week culture period for pressurized and static control samples. Data are represented as mean±standard deviation. Bars that share the same letter are not statistically different from each other. Bars that are under different letters represent statistically significant values (p<0.05, n=4). For example, a statistically significant decrease was observed from 4 wks to 8 wks in static samples (bars do not share the same letter), whereas the decreases found for pressurized samples over time was not significant (bars share the letter B).

FIG. 9 shows the total collagen per construct over the 8-week culture period. Significant increases were observed over time for both treatments. Data are represented as mean±standard deviation. Bars that share the same letter are not statistically different from each other. Bars that are under different letters represent statistically significant values (p<0.05, n=4).

FIG. 10 shows the meniscal shaped hydrogel with media and the construct being cultured in the bottom of the culture vessel.

FIG. 11 shows the meniscal shaped press used to shape the molten hydrogel in the culture vessel.

FIG. 12 shows the gross morphology of the tissue engineered constructs. Percentages given refer to the articular chondrocyte content of the culture.

FIG. 13 shows a cross-sectional view of the tissue engineered construct developed using a culture of 50% articular chondrocytes and 50% meniscal fibrochondrocytes. Red dye has been added to the image for ease of visualizing the cross section.

FIG. 14 is a graph of the wet weight of the constructs relative to the percentage of articular chondrocytes in the culture.

FIG. 15 is a graph of the percentage of water in the constructs as compared to the percentage of articular chondrocytes in the culture.

FIG. 16 is a graph of the tensile modulus of the constructs as compared to the percentage of articular chondrocytes in the culture.

FIG. 17 is a graph of the ultimate tensile strength of the constructs as compared to the percentage of articular chondrocytes in the culture.

FIG. 18 is a graph of the aggregate modulus of the constructs as compared to the percentage of articular chondrocytes in the culture.

FIG. 19 is a graph of the cell number per milligram of tissue dry weight of the constructs as compared to the percentage of articular chondrocytes in the culture.

FIG. 20 is a graph of percentage of glycosaminoglycans by dry weight of the constructs as compared to the percentage of articular chondrocytes in the culture.

FIG. 21 is a graph of percentage of collagen by dry weight of the constructs as compared to the percentage of articular chondrocytes in the culture.

FIG. 22 (A) shows a fabricated cartilage well and a tissue engineered construct press-fit into the well. This approach will be used to create an in vitro model of integration. FIG. 22 (B) shows a 50:50 co-culture made in the shape of the knee meniscus. Each hash mark is 0.5 cm.

FIG. 23 shows a negative mold comprised of agarose.

FIG. 24 shows a positive mold comprised of agarose. The agarose is saturated with culture medium, resulting in the reddish shade.

FIG. 25 shows various views of scaffoldless femur constructs made by the methods of the present disclosure.

FIG. 26 shows a comparison of the tissue engineered femur construct to a femur shaped piece of plastic. In this case, the construct was formed to resurface only part of, as opposed to the entire, femur.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure, according to certain example embodiments, is generally in the field of improved methods for tissue engineering. More particularly, the present disclosure relates to methods for forming tissue engineered constructs without the use of scaffolds and associated methods of use in tissue replacement. As used herein, a “construct” or “tissue engineered construct” refers to a three-dimensional mass having length, width, and thickness, and which comprises living mammalian tissue produced in vitro.

The methods of this disclosure generally comprise the formation of a tissue engineered constructs without the use of scaffolds or other synthetic materials. Generally, cells are seeded on a shaped hydrogel mold and allowed to self-assemble to form a construct. As used herein, “self-assemble” or “self-assembly” refers to a process in which specific local interactions and constraints between a set of components cause the components to autonomously assemble, without external assistance, into the final desired structure through exploration of alternative configurations.

Among other things, the methods of the present disclosure provide for higher cell-cell contact. Chondrocytes are unique in their need to remain in a spherical morphology to maintain their phenotype. Since the chondrocytes' only substrate for attachment is other chondrocytes in the methods of the present disclosure, this may enhance the cell to cell signaling necessary to maintain the chondrocytic phenotype. Another advantage of the methods of the present disclosure is that biocompatibility issues of the scaffold and its degradation materials are avoided as well as stress-shielding of the seeded cells by the scaffold. Cell reaction to the biomaterial, such as dedifferentiation, is also avoided. Furthermore, because stress shielding by the scaffold does not occur, the methods of the present disclosure may allow for the cells to respond directly to forces which may aid in aligning extracellular matrix production. Another advantageous feature of the present disclosure is that it allows manipulation of the thickness, geometry, and size of the resulting construct.

Formation of Shaped Hydrogel Coated Culture Vessels

The hydrogel used in conjunction with the methods of the present disclosure may comprise agarose, alignate, or combinations thereof. A “hydrogel” is a colloid in which the particles are in the external or dispersion phase and water is in the internal or dispersed phase. Suitable hydrogels are nontoxic to the cells, are non-adhesive, do not induce chondrocytic attachment, allow for the diffusion of nutrients, do not degrade significantly during culture, and are firm enough to be handled.

In particular embodiments, the hydrogel used in conjunction with the present disclosure is melted to form a molten hydrogel. The molten hydrogel is introduced into a culture vessel and may be shaped using a shaped press. The press may be shaped to accommodate the desired shape of the tissue engineered construct. In certain embodiments, the press may be in the shape of a ring. In other embodiments, the press may be a projection of, for example, the medial meniscus, femur, or kneecap rotated through 360 degrees.

The resulting pressed molten hydrogel is allowed to cool around the shape of the press. Upon removal of the press, a cooled shaped hydrogel negative mold is left remaining in the culture vessel. In certain embodiments, the shape of the resulting pressed hydrogel is a projection of the medial meniscus rotated through 360 degrees. In certain embodiments, a ring shape of the shaped hydrogel negative mold may aid in the alignment of the extracellular matrix during the formation of the tissue engineered construct by subjecting the developing construct to a hoop strain during cell culture.

The Cell Culture

The cells used in conjunction with the methods of the present disclosure may be chondrocytes or chondro-differentiated cells (referred to herein as chondrocytes), fibrochondrocytes or fibrochondro-differentiated cells (referred to herein as fibrochondrocytes), or combinations thereof. The chondrocytes may comprise articular chondrocytes. Generally, the articular chondrocytes may be from a bovine or porcine source. Alternatively if the construct is to be used for in vivo tissue replacement, the source of articular chondrocytes may be autologous cartilage from a small biopsy of the patient's own tissue, provided that the patient has healthy articular cartilage that may be used as the start of in vitro expansion. Another suitable source of chondrocytes is heterologous chondrocytes from histocompatible cartilage tissue obtained from a donor or cell line.

The fibrochondrocytes used in conjunction with the methods of the present disclosure may comprise meniscal fibrochondrocytes. Generally, the meniscal fibrochondrocytes may be from a bovine or porcine source for in vitro studies. Alternatively if the construct is to be used for in vivo tissue replacement, the source of meniscal fibrochondrocytes may be autologous fibrocartilage from a small biopsy of the patient's own tissue, provided that the patient has healthy meniscal fibrocartilage that may be used as the start of in vitro expansion. Another suitable source of fibrochondrocytes is heterologous fibrochondrocytes from histocompatible fibrocartilaginous tissue obtained from a donor or cell line.

In certain embodiments, the chondrocytes and fibrochondrocytes used in conjunction with the methods of the present disclosure may be derived from mesenchymal, embryonic, induced pluripotent stem cells, or skin cells.

The fibrochondrocytes, chondrocytes, or a co-culture of the two are suspended in media. An example of suitable media may be DMEM with 4.5 g/L-glucose and L-glutamine (Biowhittaker), 10% fetal bovine serum (Biowhittaker), 1% fungizone (Biowhittaker), 1% Penicillin/Streptomycin (Biowhittaker), 1% non-essential amino acids (Life Technologies), 0.4 mM proline (ACS Chemicals), 10 mM HEPES (Fisher Scientific), 50 μg/mL L-ascorbic acid, (Acros Organics) supplemented with 20% FBS and 10% DMSO.

In certain embodiments, the cells may comprise 50% fibrochondrocytes and 50% chondrocytes. The cells may be seeded in a shaped hydrogel negative mold or a hydrogel coated culture vessel and allowed to self-assemble. In certain embodiments, the cells may be seeded at a density in the range of about 10×10⁶ cells per cm² to 90×10⁶ cells per cm² of hydrogel coated surface. In certain embodiments, the suspension of fibrochondrocytes and chondrocytes is seeded at a density of 24×10⁶ cells/cm² of hydrogel coated surface. In other embodiments, the cells may be seeded at a density of about 29×10⁶ cells/cm² of hydrogel coated surface.

Self-Assembly of the Seeded Cells

The cells seeded on hydrogel coated culture vessels or hydrogel negative molds are allowed to self-assemble. Self-assembly may result in the formation of non-attached constructs on the hydrogel surfaces. It is preferable to use hydrogel coated surfaces instead of tissue culture treated surfaces since articular chondrocytes seeded onto standard tissue culture treated plastic (TCP) readily attach, spread, and dedifferentiate. In certain embodiments, the self-assembly process may occur in culture vessels that are shaken continuously on an orbital shaker and then pressurized. In certain embodiments, the pressurization of the cells may occur in a pressure chamber. Pressurization of the samples during the self-assembly process may aid in increased extracellular matrix synthesis and enhanced mechanical properties. In certain embodiments, the cells may be pressurized to 10 MPa at 1 Hz using a sinusoidal waveform function. In other embodiments, the cells are pressurized during culture of the self-assembled cells. In particular embodiments, a loading regimen (e.g. compressive, tensile, shear forces) may be applied to the cells during self-assembly based on physiological conditions of the native tissue in vivo. Loading of the cells during self-assembly and/or construct development may cause enhanced gene expression and protein expression in the constructs.

In particular embodiments, the cells may be treated with staurosporine, a protein kinase C inhibitor and actin disrupting agent, during the self-assembly process to reduce synthesis of αSMA, a contractile protein. Reducing αSMA in the constructs via staurosporine treatment may reduce construct contraction and may also upregulate ECM synthesis. Other anti-contraction agents can also be employed, for example, the Rho-associated kinase (ROCK) inhibitor 1, 2, Y-27632 has been shown to reduce contraction.

In other embodiments, the cells may be treated with growth factors to increase construct growth and matrix synthesis. Suitable examples of growth factors that may be used with the methods of the present disclosure include, but are not limited to, TGF-β1 and IGF-I. The dosing of the growth factors may be intermittent or continuous throughout the period of the self-assembly process. One of ordinary skill in the art, with the benefit of this disclosure, will be able to determine the appropriate dosing regimen and amount and type of growth factor to provide to the developing constructs.

Hydrogel Molds

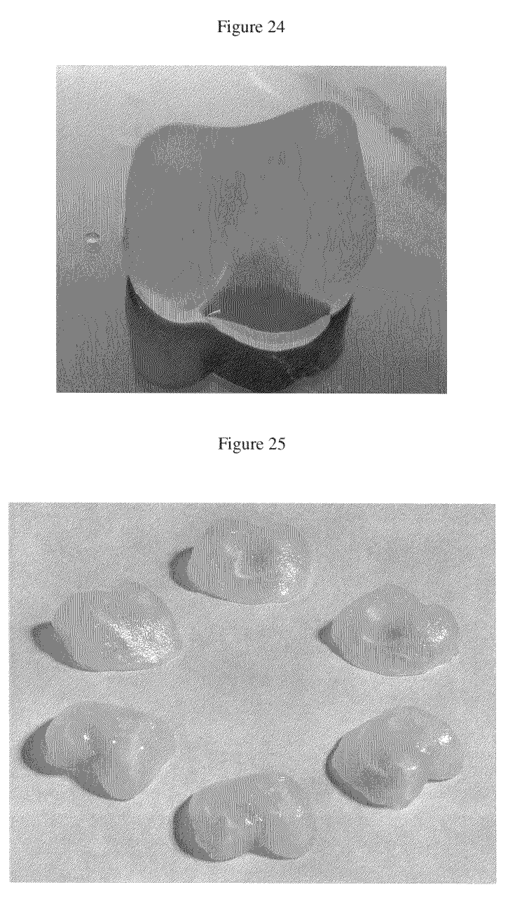

In certain embodiments, the cells used in conjunction with the methods of the present disclosure may be seeded on a hydrogel coated culture vessel and allowed to self-assemble for about 1 to about 7 days before being transferred to a shaped hydrogel negative mold. Alternatively, rather than seeding the cells on a hydrogel coated culture vessel, in certain embodiments, the cells may be seeded directly onto a shaped hydrogel negative mold. The shaped hydrogel negative mold may comprise agarose. Other non-adhesive hydrogels, for example, alignate and polyHEMA (poly 2 hydroxylthyl methacrylate), may be used in conjunction with the methods of the present disclosure. In other embodiments, the hydrogel mold may be a two piece structure comprising, a shaped hydrogel negative mold (See for example, FIG. 23) and a shaped hydrogel positive mold (See for example, FIG. 24). The shaped hydrogel negative and positive molds may comprise the same non-adhesive hydrogel or may be a comprised of different non-adhesive hydrogels. In certain embodiments, the cells may be seeded on a hydrogel coated culture vessel and allowed to self-assemble into a first construct. The first construct may be transferred to a shaped hydrogel negative mold. A shaped hydrogel positive mold may be applied to the negative mold to form a mold-construct assembly. The mold-construct assembly may then further be cultured to form a second construct. As used herein, the term “mold-construct assembly” refers to a system comprising a construct or cells within a shaped positive and a shaped negative hydrogel mold.

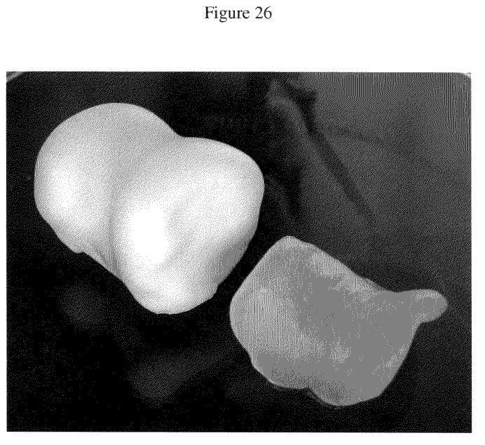

In certain embodiments, the molds may be shaped from a 3-D scanning of a total joint to result in a mold fashioned in the shape of said joint. In other embodiments, the molds may be shaped from a 3-D scanning of the ear, nose, or other non-articular cartilage to form molds in the shapes of these cartilages. In certain embodiments, the mold may be shaped to be the same size as the final cartilaginous product. In other embodiments, the molds may be shaped to be smaller than the final cartilaginous product. In certain embodiments, the molds may be fashioned to a portion of a joint or cartilage so that it serves as a replacement for only a portion of said joint or cartilage (See FIGS. 25 and 26).

Analysis of the Constructs

The properties of the constructs may be tested using any number of criteria including, but not limited to, morphological, biochemical, and biomechanical properties, which also may be compared to native tissue levels. In this context, morphological examination includes histology using safranin-O and fast green staining for glycosaminoglycan (GAG) content, as well as picro-sirius red staining for total collagen, immunohistochemistry for collagens I and II, and confocal and scanning electron microscopies for assessing cell-matrix interactions. Biochemical assessments includes picogreen for quantifying DNA content, DMMB for quantifying GAG content, hydroxyproline assay for quantifying total collagen content, and ELISA for quantifying amounts of specific collagens (I and II).

Constructs also may be evaluated using one or more of incremental tensile stress relaxation incremental compressive stress relaxation, and biphasic creep indentation testing to obtain moduli, strengths, and viscoelastic properties of the constructs. Incremental compressive testing under stress relaxation conditions may be used to measure a construct's compressive strength and stiffness. Incremental tensile stress relaxation testing may be used to measure a construct's tensile strength and stiffness. Additionally, indentation testing under creep conditions may be used to measure a construct's modulus, Poisson's ratio, and permeability.

Without wishing to be bound by theory or mechanism, although both collagen II and GAGs are excellent predictors of biomechanical indices of cartilage regeneration, typically only collagen II exhibits a positive correlation. Though seemingly this hypothesis is counterintuitive for compressive properties, as GAG content is usually thought to correlate positively with compressive stiffness, our results show that in self-assembled constructs, GAG is negatively correlated with the aggregate modulus (R²=0.99), while collagen II is positively correlated (R²=1.00).

The constructs of the present disclosure may be assessed morphologically and/or quantitatively. Quantitatively, the constructs of the present disclosure may be evaluated using a functionality index (FI) as described in Eq. 1. The functionality index is an equally weighted analysis of ECM production and biomechanical properties that includes quantitative results corresponding to the constructs' salient compositional characteristics (i.e., amounts of collagen II and GAG) and biomechanical properties (compressive and tensile moduli and strengths).

$$F I = \frac{1}{4} ( \begin{matrix}( 1 - \frac{( {G}_{nat} - {G}_{sac} )}{{G}_{nat}} ) + ( 1 - \frac{( {C}_{nat} - {C}_{sac} )}{{C}_{nat}} ) + \\ \frac{1}{2} ( 1 - \frac{( {E}_{nat}^{T} - {E}_{sac}^{T} )}{{E}_{nat}^{T}} ) + \frac{1}{2} ( 1 - \frac{( {E}_{nat}^{C} - {E}_{sac}^{C} )}{{E}_{nat}^{C}} ) + \\ \frac{1}{2} ( 1 - \frac{( {S}_{nat}^{T} - {S}_{sac}^{T} )}{{S}_{nat}^{T}} ) + \frac{1}{2} ( 1 - \frac{( {S}_{nat}^{C} - {S}_{sac}^{C} )}{{S}_{nat}^{C}} )\end{matrix} ) \qquad Eq . ( 1 )$$

In this equation, G represents the GAG content per wet weight, C represents the collagen II content per wet weight, E^{T} represents the tensile stiffness modulus, E^{C} represents the compressive stiffness modulus, S^{T} represents the tensile strength, and S^{C} represents the compressive strength. Each term is weighted to give equal contribution to collagen, GAG, tension, and compression properties. The subscripts nat and sac are used to denote native and self-assembled construct values, respectively. The aggregate modulus is not used in Eq. 1, as it is expected to mirror the compressive modulus obtained from incremental compressive stress relaxation. Similarly, the amount of collagen I is not be used in Eq. 1, as this type of collagen may not appear in a measurable fashion; however, if the amount of collagen I is non-negligible, FI may be altered accordingly to account for it.

Each term grouped in parentheses in Eq. 1 calculates how close each construct property is with respect to native values, such that scores approaching 1 denote values close to native tissue properties. Equal weight is given to GAG, collagen II, stiffness (equally weighted between compression and tension), and strength (also equally weighted between compression and tension). This index, FI, will be used to assess the quality of the construct compared to native tissue values, with a lower limit of 0 and an unbounded upper limit, with a value of 1 being a construct possessing properties of native tissue. However, the FI can exceed 1 if optimization results in constructs of properties superior to native tissue.

Methods of Using the Tissue Engineered Constructs

A hydrogel coated culture vessel or shaped hydrogel negative mold is seeded with cells to produce new tissue, such as tissue of the knee meniscus, tendons, and ligaments. The hydrogel coated culture vessel or shaped hydrogel negative mold is typically seeded with cells; the cells are allowed to self-assemble to form a tissue engineered construct. In certain embodiments, applications of the tissue engineered construct include the replacement of tissues, such as the knee meniscus, joint linings, the temporomandibular joint disc, tendons, or ligaments.

The constructs may be treated with collagenase, chondroitinase ABC, and BAPN to aid in the integration of the constructs with native, healthy cartilage surrounding the desired location of implantation. The integration capacity of a construct with native tissue is crucial to regeneration. A wound is naturally anti-adhesive, but debridement with chondroitinase ABC and/or collagenase removes anti-adhesive GAGs and enhances cell migration by removing dense collagen at the wound edge. BAPN, a lysyl oxidase inhibitor, may cause the accumulations of matrix crosslinkers and may, thus, strengthen the interface between the construct and native tissue at the desired location of implantation.

The tissue engineered constructs may be implanted into a subject and used to treat a subject in need of tissue replacement. In certain embodiments, the constructs may be grown in graded sizes (e.g. small, medium, and large) so as to provide a resource for off-the-shelf tissue replacement. In certain embodiments, the constructs may be formed to be of custom shape and thickness. In other embodiments, the constructs may be devitalized prior to implantation into a subject.

To facilitate a better understanding of the present disclosure, the following examples of specific embodiments are given. In no way should the following examples be read to limit or define the entire scope of the disclosure.

### EXAMPLES

### Isolation and Seeding of Chondrocytes and Fibrochondrocytes

Chondrocytes were isolated from the distal femur of week-old male calves (Research 87 Inc.) less than 36 hrs after slaughter, with collagenase type I (Worthington) in culture medium. The medium was DMEM with 4.5 g/L-glucose and L-glutamine (Biowhittaker), 10% fetal bovine serum (Biowhittaker), 1% fungizone (Biowhittaker), 1% Penicillin/Streptomycin (Biowhittaker), 1% non-essential amino acids (Life Technologies), 0.4 mM proline (ACS Chemicals), 10 mM HEPES (Fisher Scientific), and 50 μg/mL L-ascorbic acid (Acros Organics). Chondrocytes were frozen in culture medium supplemented with 20% FBS and 10% DMSO at −80° C. for 2 wks to a month before cells from two donor legs were pooled together. Cells from each leg were counted on a hemocytometer, and viability was assessed using a trypan blue exclusion test. Each leg yielded roughly 150 million cells, and viability was greater than 99% for both legs. After thawing, viability remained greater than 92%.

Fibrochondrocytes were harvested from the medial meniscus of approximately 1-wk old male calves (Research 87, Boston, Mass.) less than 36 hrs after slaughter, with collagenase in the culture medium. The medium was DMEM with 4.5 g/L-glucose and L-glutamine, 10% FBS, 1% fungizone, 1% Penicillin/Streptomycin, 1% non-essential amino acids, 0.4 mM proline, 10 mM HEPES, and 50 μg/mL L-ascorbic acid. Cells were frozen at −80° C. in culture medium supplemented with 20% FBS and 10% DMSO for 2 to 4 wks before cells from donor legs can be pooled together.

Formation of the Hydrogel Molds.

A silicon positive die consisting of 5 mm diameter×10 mm long cylindrical prongs has been constructed to fit into a 6-well plate. To construct the agarose mold, sterile, molten 2% agarose will be introduced into a well fitted with the silicon positive die. The agarose will be allowed to gel at room temperature for 15 min. The agarose mold will then be separated from the silicon positive die and submerged into two exchanges of culture medium. The agarose mold will thus be completely saturated with the culture medium by the time of cell seeding. To each agarose well, 5.5×10⁶ cells will be added in 50 μl of culture medium. The cells will self-assemble within 24 hrs in the agarose wells and will be maintained in the same wells for 3 days. These self-assembled constructs will then be placed into larger agarose wells with 3 mL of medium, exchanged once every 3 days. Constructs will be cultured for the specified amount of time; t=0 will be defined as 24 hrs after seeding.

Self Assembly and Culture of the Tissue engineered Constructs.

Each well of a 96 well plate was coated with 100 μl of 2% molecular biology grade agarose (Sigma). The plates were tilted to spread the agarose along the walls, and then inverted to shake out the excess agarose. To each well, 5.5 million chondrocytes in 300 μl of culture medium were introduced. Within 24 hrs, the cells formed non-attached constructs at the bottom of each well, and these constructs were maintained in the 96 well plates for 4 wks before being transferred to agarose coated 46 well plates. Each day 500 μl medium was changed (250 μl twice daily). Time zero (t=0) was defined as 24 hrs after seeding.

Self assembly on tissue culture treated plates without hydrogel coating was also assessed. To each well of a 96-well TCP plate, 5.5 million cells in 300 μl of culture medium were introduced. Within 24 hrs, the cells formed attached constructs at the bottom of each well, and these constructs were maintained in the 96 well plates for 4 wks, and were then transferred to tissue culture treated 46-well plates. Each day 500 μl medium was changed (250 μl twice daily). Time zero (t=0) was defined as 24 hrs after seeding.

After seeding chondrocytes on either TCP or over an agarose substratum, the chondrocytes formed cohesive constructs within 24 hrs (defined as t=0 wks). At t=0 wks, the constructs could be manipulated in the medium but were not testable mechanically. Thus, histological, biochemical, and biomechanical data were collected at t=4 wks and 8 wks. Since constructs cultured over agarose consistently outperformed constructs cultured on TCP in terms of biochemistry and biomechanics (significance defined as p<0.05), they merited an extended culture period to t=12 wks.

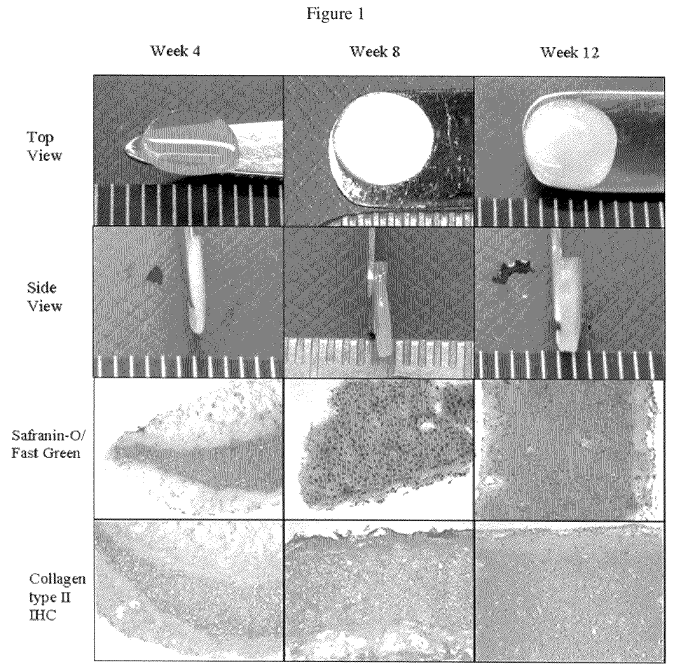

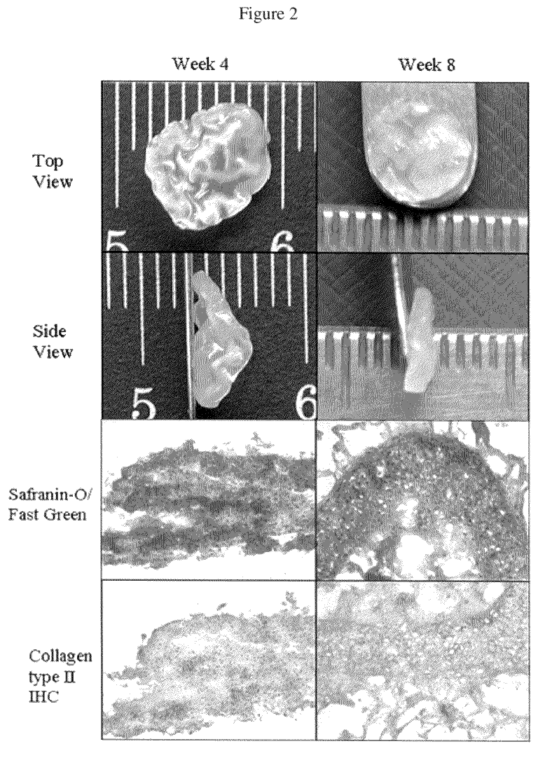

Constructs from both treatments increased in opacity over time. After 24 hrs, cells on agarose formed one cohesive nodule that was not attached to the substratum. Other than the single nodule, the agarose surface did not have any other attached cells or nodules. In contrast, the control cells readily attached to the bottom of the TCP wells and formed nodules that adhered to TCP and detached from the constructs as time progressed. Constructs cultured over agarose appeared smooth, flat, and hyaline-like in appearance. Disks 6-mm in diameter were punched out of the center of the constructs cultured over agarose for mechanical testing, and these are shown in FIG. 1, rows 1 and 2. Unlike the constructs formed over agarose, the constructs cultured on TCP became contorted with folds (FIG. 2).

The constructs cultured over agarose assumed a bowl shape and increased in diameter from an initial 5 mm to more than 1 cm at t=12 wks. The thickness of constructs also increased significantly over time, from 460±78 μm at t=4 wks, to 770±75 μm at t=8 wks, and to 950±80 μm at t=12 wks. The constructs were not of uniform thickness throughout, and the thickness of the thinnest portion (the 6-mm punched out disks) is reported, since this was the mechanically tested region. The constructs cultured on TCP also significantly increased in thickness over time, from 344±39 μm at t=4 wks to 663±34 μm at t=8 wks. The constructs formed over agarose were significantly thicker than those formed over TCP for these time points.

The constructs cultured over agarose displayed many similarities to native tissue. Even within the loose shell (FIG. 1), the chondrocytes were in lacunae, as compared to the TCP constructs, where the loose shell contained no lacunae and fewer cells. In addition, cells appeared to rest in lacunae that were mostly elongated in the z-direction (thickness) for constructs formed over agarose at t=4 wks. Where more than one cell rested in the same lacuna, these cells were also stacked in the z-direction. The curl of the bowl-shaped constructs suggests a pre-tensed state. At t=4 wks, many lacunae were aligned in the z-direction, which may indicate an organization (FIG. 1) yet to be reported by studies using scaffolds. The constructs formed over agarose showed increases in staining intensity and coverage for safranin-O from t=4 wks to t=8 wks. Over this time the constructs also matured as to be devoid of the loosely organized shell. On TCP, however, the cells did not appear to organize in any particular direction, and staining intensity did not increase over time.

Histology and Immunohistochemistry of the Tissue Engineered Constructs.

Samples were frozen and sectioned at 14 μm. Safranin-O and fast green staining were used to examine GAG distribution. Slides were also processed with IRC to test for the presence of collagen type I (COL1) and collagen type II (COL2) on a Biogenex i6000 autostainer. After fixing in chilled acetone, the slides were rinsed with IRC buffer (Biogenex), quenched of peroxidase activity with hydrogen peroxide/methanol, and blocked with horse serum (Vectastain ABC kit). The slides were then incubated with either mouse anti-COL1 (Accurate Chemicals) or mouse anti-COL2 (Chondrex) antibodies. The secondary antibody (mouse IgG, Vectastain ABC kit) was then applied, and color was developed using the Vectastain ABC reagent and DAB (Vector Laboratories).

To assess DNA content, GAG content, and collagen content, samples were digested with 125 μg/mL papain (Sigma) in 50 mM phosphate buffer (pH=6.5) containing 2 mM N-acetyl cysteine (Sigma) and 2 mM EDT A (Sigma) at 65° C. overnight. Total DNA content was measured by Picogreen® Cell Proliferation Assay Kit (Molecular Probes). Total sulfated GAG was then quantified using the Blyscan Glycosaminoglycan Assay kit (Biocolor), based on 1,9-dimethylmethylene blue binding. After being hydrolyzed by 2 N NaOH for 20 min at 110° C., samples were assayed for total collagen content by a chloramine-T hydroxyproline assay.

For constructs cultured over agarose, at t=4 wks the constructs displayed two distinct regions (FIG. 1, rows 3 and 4). A porous, diffuse outer shell with low safranin-O and low collagen type II staining, indicating low amounts of GAG and collagen in this area. In contrast to the positive collagen type II staining, collagen type I staining was not observed at any time. A protein gel (data not shown) further confirmed the presence of collagen type II alpha 1 chains and the absence of collagen type I alpha 1 and alpha 2 chains, indicating maintenance of the chondrocytic phenotype. As a control, sections stained with safranin-O/fast green for GAG and with Immunohistochemistry for collagen type II on constructs cultured over TCP were used. As with t=4 wks constructs cultured over agarose, at t=8 wks, a loosely organized shell was seen around the constructs cultured on TCP. Matrix from this shell easily peeled off as sheets. This was in stark contrast to the cohesive constructs formed over agarose.

Quantitative Biochemistry of the Constructs.

Samples (self assembled without pressurization) were digested with 125 μg/mL papain (Sigma) in 50 mM phosphate buffer (pH=6.5) containing 2 mM N-acetyl cysteine (Sigma) and 2 mM EDTA (Sigma) at 65° C. overnight. Total DNA content was measured by Picogreen® Cell Proliferation Assay Kit (Molecular Probes). Total sulfated GAG was then quantified using the Blyscan Glycosaminoglycan Assay kit (Biocolor), based on 1,9-dimethylmethylene blue binding. After being hydrolyzed by 2 N NaOH for 20 min at 110° C., samples were assayed for total collagen content by a chloramine-T hydroxyproline assay.

Constructs cultured over agarose gained mass over the culture period, and, at each time point, these constructs contained significantly larger mass than constructs cultured on TCP. Wet weights (ww) for constructs cultured over agarose were 39.1±4.3 mg at t=4 wks, 53.1±4.2 mg at t=8 wks, and 99.0±5.7 mg at t=12 wks. The ww of TCP constructs increased from 28.1±3.1 mg at t=4 wks to 39.1±4.3 mg at t=8 wks. For the constructs formed over agarose, the total cell number did not show significant changes during the culture period and ranged from 5.8±1.2 to 7.1±1.2 million per construct. The number of cells in these constructs was more, though not significantly, than the constructs cultured over TCP, which showed an increase in cell number from 4.5±1.2 to 6.3±1.4 million cells per construct over the culture period.

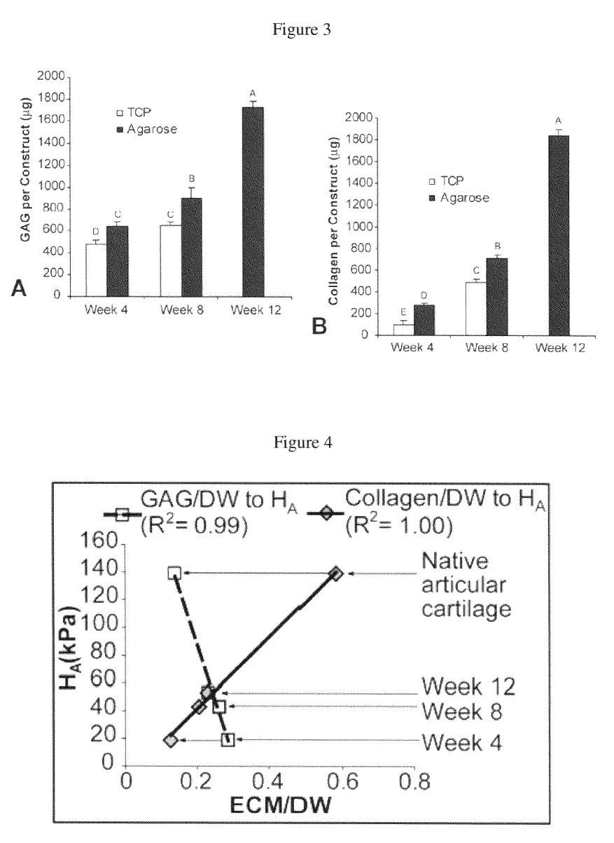

The constructs formed over agarose contained significantly higher GAG and collagen per sample at each time point when compared to control (FIG. 3). For constructs cultured over agarose, the total GAG per sample increased significantly from t=4 wks at 640±100 μg to 1700±210 μg at t=12 wks (FIG. 3A). Total GAG per construct also increased significantly for constructs cultured on TCP, from 480±40 μg at t=4 wks to 650±60 μg at t=8 wks (FIG. 3A). The total collagen per construct cultured over agarose significantly increased from 280±40 μg at t=4 wks to 1840±170 μg at t=12 wks (FIG. 3B). For constructs cultured on TCP, total collagen per construct increased significantly from 93±16 μg at t=4 wks to 480±50 μg at t=8 wks (FIG. 3B).

To compare the ECM produced in the constructs to bovine articular cartilage (BAC), the biochemical data were normalized to dry weight (dw). Both treatments produced significantly more GAG/dw at all time points compared to BAC. GAG/dw of agarose constructs displayed a decreasing trend with time, from 0.29±0.05 (g GAG/g construct) at t=4 wks, to 0.26±0.03 at t=8 wks, to 0.23±0.03 at t=12 wks. Collagen/dw increased from 0.13±0.04 at t=4 wks, to 0.21±0.02 at t=8 wks, to 0.23±0.03 at t=12 wks. At t=12 wks, GAG/dw of construct formed over agarose was ⅔ higher than BAC, while collagen/dw reached more than ⅓ the level of BAC (FIG. 4). Collagen/dw provided a strong positive correlation (R²=1.00) to HA values (FIG. 4), and may serve as an excellent predictor of construct stiffness.

Mechanical Analysis of the Constructs.

For mechanical analysis, samples were evaluated with an automated indentation apparatus. Each specimen was attached to the sample holder by use of cyanoacrylate glue, and was submerged in saline solution. The specimen was positioned under the load shaft of the apparatus so that the sample surface test point was perpendicular to the indenter tip. The specimen was automatically loaded with a tare mass of 0.4 g (0.004 N), using a 1.67 mm-diameter rigid, flat-ended, porous indenter tip. Samples were allowed to reach tare creep equilibrium, which was defined as deformation <10⁻⁶ mm/s or a maximum creep time of 10 min. When tare equilibrium was reached, a step mass of 2.34 g (0.023 N) was applied. Displacement of the sample surface was measured until equilibrium was reached or a maximum creep time of 1.5 hrs elapsed. At that time, the step load was removed, and the displacement was recorded until equilibrium was again reached. Preliminary estimations of the Young's modulus of the samples were obtained using the analytical solution for the axisymmetric Boussinesq problem with Papkovich potential functions. The intrinsic mechanical properties of the samples were then determined using the linear biphasic theory. Calf tissue from the tibial plateau was tested to yield an HA of 139±41 kPa (n=5).

Constructs from both groups were not mechanically testable at t=0 wks. Starting from t=4 wks, constructs from both treatments were tested biomechanically under conditions of creep indentation. Constructs cultured over agarose consistently outperformed constructs cultured over TCP.

For constructs cultured over agarose, Boussinesq-Papkovich estimates of the Young's modulus ranged from 70-75 kPa at t=4 wks, 65-101 kPa at t=8 wks and 78-121 kPa at t=12 wks. Boussinesq-Papkovich estimates of the Young's modulus for constructs cultured over TCP ranged from 39-61 kPa at t=4 wks, and did not significantly increase by t=8 wks. Using the biphasic theory, the aggregate modulus (HA) of the t=4 wks constructs formed over agarose was 19±3 kPa, and this significantly increased to 43±13 kPa at t=8 wks. “Aggregate modulus” is a conventional measurement used in characterizing cartilage. Ultimately, the samples reached an HA of 53±9 kPa after 12 wks (See Table 1 below). Control constructs were significantly softer at each time point, ranging from 13±4 kPa at t=4 wks to 19±3 kPa at t=8 wks (See Table 1 below). The permeability and Poisson's ratio values were not significantly different across the two treatments. At t=8 wks, constructs cultured on TCP reached 14% of the stiffness of calf articular cartilage, whereas constructs on agarose reached 31%. By t=12 wks, constructs cultured over agarose increased their stiffness to almost 40% of the stiffness of native tissue. By t=8 wks, the permeability values of constructs cultured on TCP and over agarose were not significantly different from native tissue. The Poisson's ratios of constructs from both groups were initially greater than native tissue, though these values decreased over time to approach native tissue. The results of mechanical analysis can be seen in Table 1 below.

TABLE 1
Results of Mechanical Analysis

| | H_{A} (kPa) | k (10⁻¹⁵ m⁴/Ns) | ν |
|---|---|---|---|
| Week 4, over agarose | 19 ± 3 | 17 ± 6 | 0.23 ± 0.08 |
| Week 8, over agarose | 43 ± 13 | 40 ± 21 | 0.11 ± 0.08 |
| Week 12, over agarose | 53 ± 9 | 22 ± 24 | 0.03 ± 0.05 |
| Week 4, on TCP | 13 ± 4 | 24 ± 10 | 0.22 ± 0.11 |
| Week 8. on TCP | 19 ± 3 | 33 ± 21 | 0.07 ± 0.09 |
| Native articular cartilage | 139 ± 41 | 42 ± 28 | 0.01 ± 0.01 |

The strong correlations of HA to ECM/dw are linear and, as shown in FIG. 4, are in a linear relationship to native tissue values. This is an exciting finding as it suggests that the tissue produced in this study develops in a manner analogous to native articular cartilage. Extended culture periods, bioactive agents, or mechanical stimuli may aid this tissue to further progress down this pathway towards native tissue-like functionality.

The results of the above examples comparing hydrogel coated and TCP surfaces show that, indeed, chondrocytes attached and flattened onto the TCP. In addition, constructs formed over agarose were smooth in appearance, thicker, contained more ECM, and were stiffer than those formed on TCP. When seeded on TCP, cells formed numerous distinct nodules that did not contribute in forming one uniform cohesive construct. In contrast, cells on agarose did not spread, but rather self-assembled immediately into one large nodule that increased in diameter and thickness over time. The self-assembled cartilage construct formed over agarose contained spherical cells with a chondrocytic phenotype. This tissue engineered product also contained ⅔ more GAG/dw than native tissue. Collagen/dw reached ⅓ the level of native tissue, and the stiffness reached more than ⅓ that of native tissue. Based on these observations, it is suggested that the scaffold-free self-assembling process over an agarose substratum may provide a feasible culture methodology to produce functionally relevant tissue analogues. Further experimentation involving pressurization of the samples during self assembly were then performed on hydrogel coated surfaces.

Self Assembly with Pressurization.

Agarose molds were constructed out of agarose with 3 mm diameter wells. To each well, 5.1 million chondrocytes were seeded and allowed to self-assemble. After self-assembling for 24 hr, defined as t=0 wk, the constructs were transferred to agarose coated 100 mm diameter petri dishes. An equivalent of 3 mL of medium was exchanged per construct every 2 days. The petri dishes were shaken continuously on an orbital shaker at 60 rpm beginning at t=0. At t=2 wk of culture, constructs were divided into pressure and control groups.

Both control and pressure group constructs were loaded into heat sealable bags (Kapak) previously sterilized by ethylene oxide. To each bag, medium was added, and the bags were tapped gently to release any residual bubbles adhering to the bottom of the bag. The bags were heat-sealed without any bubbles inside.

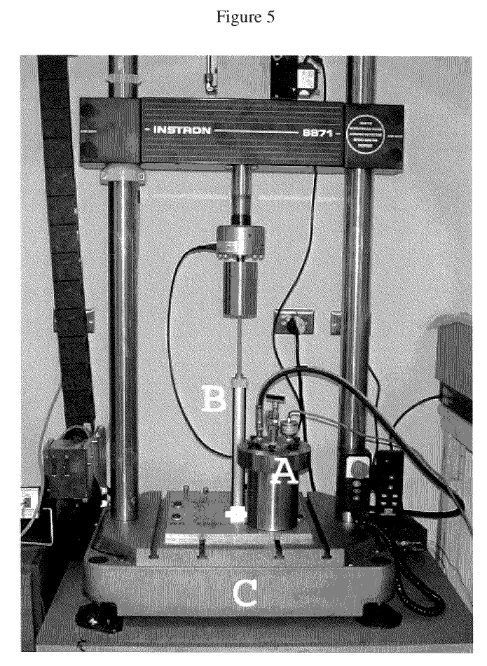

Control specimens were placed into an opened pressure chamber, while pressure specimens were placed into a pressure chamber (Parr Instrument Company), filled with water, and sealed underwater without any bubbles inside. The pressure chamber is a 1.2 L stainless-steel vessel capable of withstanding pressures upwards of 13 MPa (FIG. 5, A). It is connected to a water-driven piston (PHD Inc.) (FIG. 5, B) via a stainless-steel ¼″ hose (Dunlop) rated for pressures up to 40 MPa. The piston is connected to an Instron 8871 (FIG. 5, C), controlled using the Instron WaveMaker software. For 5 consecutive days a week, the specimens were pressurized to 10 MPa at 1 Hz using a sinusoidal waveform for 4 hrs. After the execution of the desired regimen, the pressure chamber was disassembled, and the pouches were sterilized with 70% ethanol. In a sterile culture hood, the pouches were opened with autoclaved instruments and the samples were then returned to orbitally shaken culture dishes.

The pressure set-up assembled in this study applied intermittent hydrostatic pressure at 10 MPa, 1 Hz, 4 hrs a day consistently over an 8-week period. Articular chondrocyte constructs subjected to this loading regimen were shown to withstand the repeated mechanical stimulus.

At t=4 wks and t=8 wks, samples from both pressurized and controls were frozen and sectioned at 14 μm. Safranin-O and fast green staining were used to examine GAG distribution. Slides were also processed by immunohistochemistry to test for the presence of collagen type II (COL2) on a Biogenex i6000 autostainer. After fixing in 4° C. acetone, the slides were rinsed with immunohistochemistry buffer (Biogenex), quenched of peroxidase activity with hydrogen peroxide/methanol, and blocked with equine serum (Vectastain ABC kit). The slides were then incubated with either mouse anti-collagen type I antibody (Accurate Chemicals) at 1:1500 dilution in PBS or mouse anti-collagen type II antibody (Chondrex) at 1:1000 dilution on PBS. The secondary antibody (antimouse IgG, Vectastain ABC kit) was then applied, and color was developed using the Vectastain ABC reagent and DAB (Vector Laboratories). Slides stained with mouse IgG 1/2a/2b (Accurate Chemicals) served as negative controls.

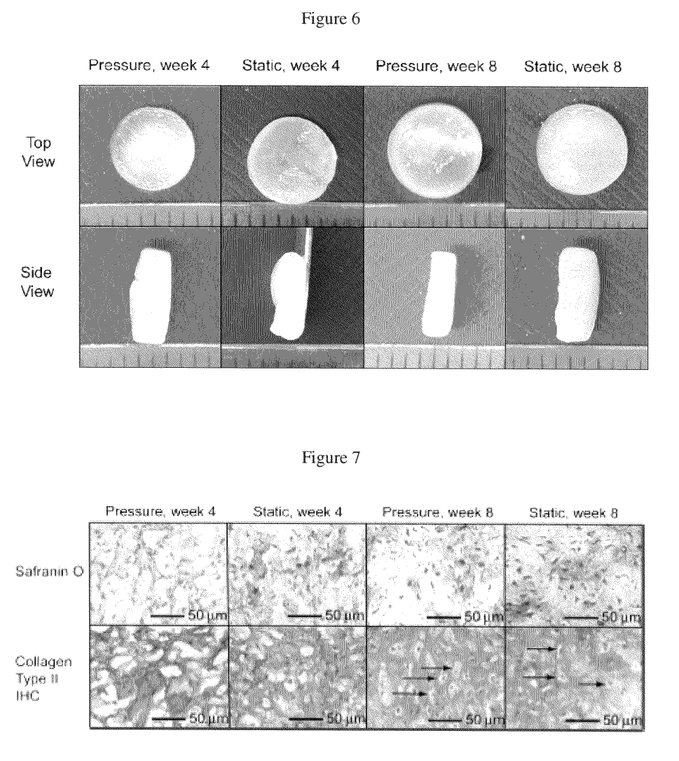

The gross appearance of the 3-D culture is shown in FIG. 6. After 4 wks of culture, the pressurized samples reached thicknesses of 2.01±0.04 mm. Likewise, the controls reached thicknesses of 1.98±0.51 mm. The thicknesses of the constructs were maintained for the remainder of the culture period, and did not differ significantly between treatments. By t=8 wks, both pressurized and control constructs stained positive for collagen type II throughout the thickness of the construct. Safranin O staining for GAG was also observed throughout the constructs (FIG. 7). At this time, the cells were round and rested in lacunae (FIG. 7, arrows).

Quantitative Biochemistry of the Constructs after Pressurization.

Samples (self assembled with pressurization) were digested with 125 μg/mL papain (Sigma) in 50 mM phosphate buffer (pH=6.5) containing 2 mM N-acetyl cysteine (Sigma) and 2 mM EDTA (Sigma) at 65° C. overnight. Total DNA content was measured by Picogreen® Cell Proliferation Assay Kit (Molecular Probes). Total sulfated GAG was then quantified using the Blyscan Glycosaminoglycan Assay kit (Biocolor), based on 1,9-dimethylmethylene blue binding. After being hydrolyzed by 2 N NaOH for 20 min at 110° C., samples were assayed for total collagen content by a chloramine-T hydroxyproline assay.

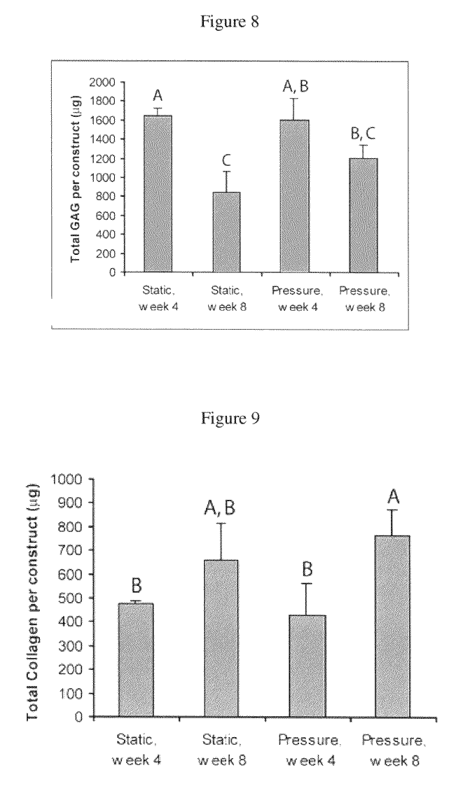

By 4 wks, pressurized constructs reached a wet weight (WW) of 87.5±7.5 mg, and the wet weight remained steady, reaching 92.7±9.0 mg at 8 wks. The same WW range was observed with control samples. Control sample WW was 92.3±5.6 mg at 4 wks and 83.9±11.7 mg at 8 wks. This decrease was not statistically significant. Total GAG per construct significantly decreased in the control samples, while the pressurized samples showed an insignificant decrease (FIG. 8). For the pressurized samples, GAG content decreased from 1590±230 μg at t=4 wks to 1200±140 μg at t=8 wks (FIG. 8), though this decrease was not significant. GAG per construct for the control decreased significantly from 1600±80 μg at t=4 wks to 840±220 μg at t=8 wks (FIG. 8). Total collagen content increased significantly for pressurized samples only from 430±130 μg at t=4 wks to 770±100 μg at t=8 wks (FIG. 9). Collagen per construct for the control also increased from 430±130 μg at t=4 wks to 660±150 μg at t=8 wks (FIG. 9), though this increase was not significant.

For pressurized samples, GAG/DW decreased from 31%±5% at t=4 to 28%±2% at t=8 wks, though this decrease was not significant. Collagen/DW increased significantly from 8%±1% at t=4 wks to 17%±4% at t=8 wks. GAG/DW observed in controls was 47%±19% at t=4 wks and 22%±6% at t=8 wks. This significant decrease was not observed in the pressurized samples. Collagen/DW of constructs observed in controls was 14%±6% at t=4 wks and 17%±4% at t=8 wks, and this increase was not significant.

In the pressurized samples, the total number of cells per construct was found to increase from 3.6±0.8 million at t=4 wks to 4.5±1.3 million at t=8 wks. Total cell numbers in controls ranged from 4.1±2.7 million at t=4 wks to 4.2±1.2 million at t=8 wks in controls. The number of cells at t=4 wks were significantly fewer than the 5.1±0.1 million seeded, as cell loss was observed in orbital culture.

Mechanical Analysis of the Constructs following Pressurization.

For mechanical analysis, samples were evaluated with an automated indentation apparatus. Each specimen was attached to the sample holder by use of cyanoacrylate glue, and was submerged in saline solution. The specimen was positioned under the load shaft of the apparatus so that the sample surface test point was perpendicular to the indenter tip. The specimen was automatically loaded with a tare mass of 0.4 g (0.004 N), using a 1.67 mm-diameter rigid, flat-ended, porous indenter tip. Samples were allowed to reach tare creep equilibrium, which was defined as deformation <10⁻⁶ mm/s or a maximum creep time of 10 min. When tare equilibrium was reached, a step mass of 2.34 g (0.023 N) was applied. Displacement of the sample surface was measured until equilibrium was reached or a maximum creep time of 1.5 hrs elapsed. At that time, the step load was removed, and the displacement was recorded until equilibrium was again reached. Preliminary estimations of the Young's modulus of the samples were obtained using the analytical solution for the axisymmetric Boussinesq problem with Papkovich potential functions. The intrinsic mechanical properties of the samples were then determined using the linear biphasic theory. Calf tissue from the tibial plateau was tested to yield an HA of 139±41 kPa (n=5).

The aggregate modulus of pressurized samples reached 20±5 kPa at t=4 wks and maintained this level to the end of the culture period. The stiffness of the controls was not significantly different, reaching 22±7 kPa at t=4 wks. As with pressurized samples, the stiffness of the controls also remained constant to t=8 wks. Permeability of the samples at t=4 wks ranged from 10±2 (10⁻¹⁵) m⁴/Ns in pressurized samples to 13±6 (10⁻¹⁵) m⁴/Ns in controls. The permeability of the samples also remained constant throughout the culture period. The Poisson's ratio values of constructs ranged from 0.006 to 0.015 across treatments and were not significantly different over culture time.

The previous examples involving pressurization show, for the first time, that long-term culture of tissue engineered articular cartilage construct benefits from intermittent hydrostatic pressure and positively affects ECM synthesis in the chondrocyte constructs. Although the specific loading regimen applied the aforementioned examples did not result in improved mechanical properties over the control, such differences may manifest themselves over time.

Formation and Analysis of the Shaped Constructs.

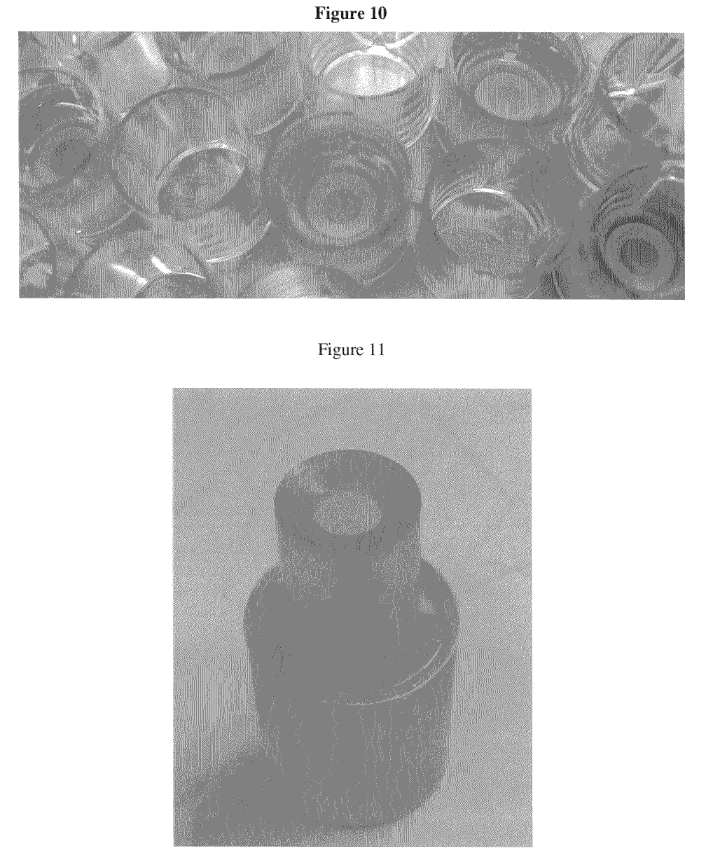

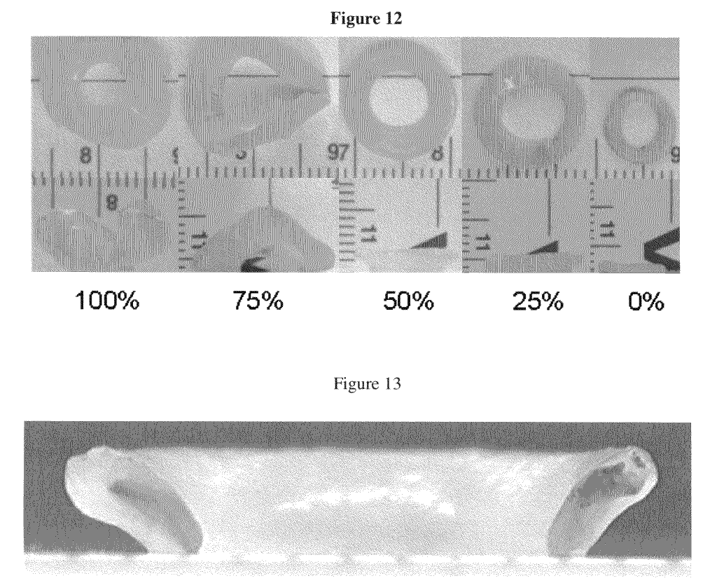

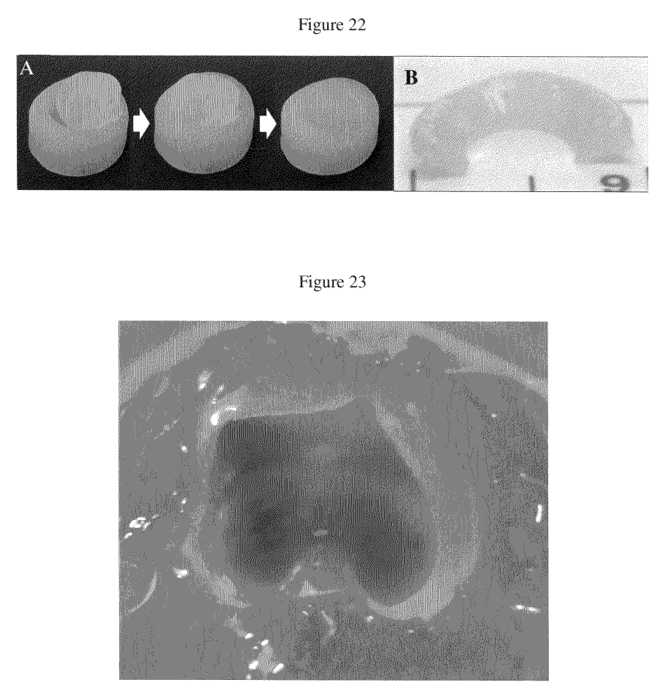

Cell suspensions were seeded on the cooled, pressed hydrogel coated surfaces. See FIG. 10, FIG. 11, and FIG. 12. 100% articular chondrocytes and 100% meniscal fibrochondrocytes were seeded on the hydrogel coated surfaces. Co-cultures of the two were also seeded comprising: 75% articular chondrocytes and 25% meniscal fibrochondrocytes, 50% articular chondrocytes and 50% meniscal fibrochondrocytes, and 25% articular chondrocytes and 75% meniscal fibrochondrocytes. FIG. 13 is an image of the developing shaped construct. See also FIG. 22.

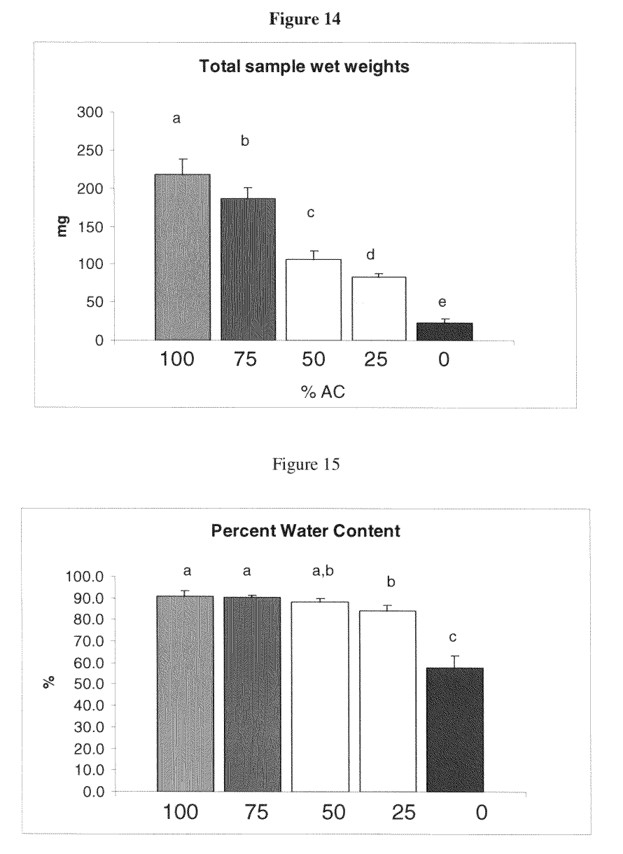

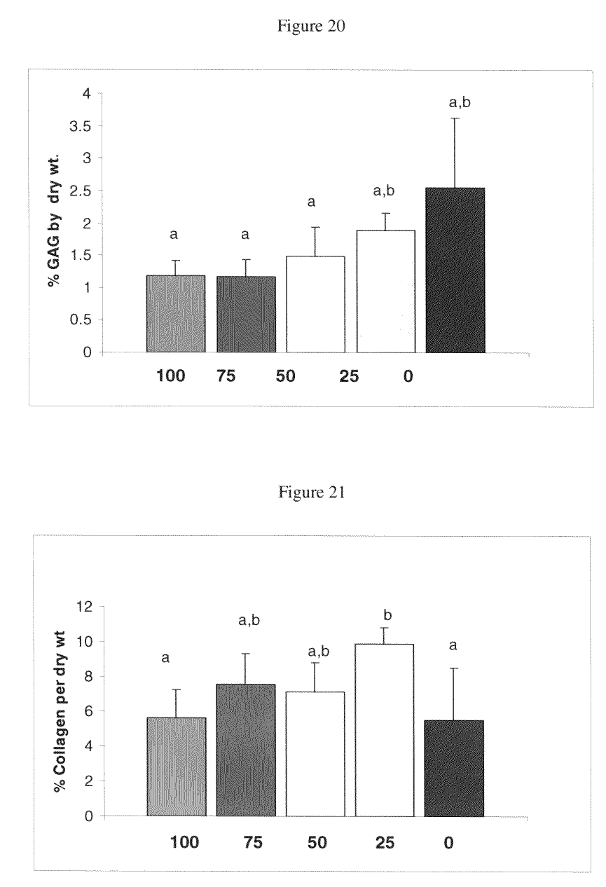

Quantitative biochemical analysis indicates that glycosaminoglycans and collagen type II were present in all of the constructs. (FIG. 20 and FIG. 21). In addition, wet weight and dry weight compositions of the constructs were analyzed. See FIG. 14, FIG. 15, and FIG. 19.

Mechanical Analysis of the Constructs.

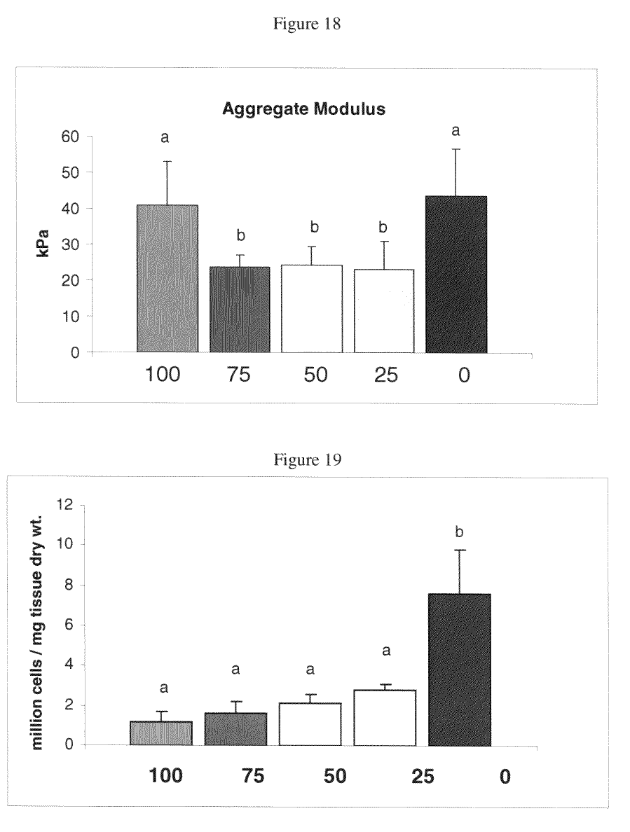

Mechanical testing of the representative constructs formed from different co-culture compositions was performed. All constructs formed from co-cultures had a lower aggregate modulus than either the construct formed from 100% articular chondrocytes or the construct formed from 100% meniscal fibrochondrocytes (See FIG. 18).

The tensile modulus of the developing constructs were analyzed using known techniques. The tensile modulus appears to increase with increasing fibrochondrocyte composition (See FIG. 17).

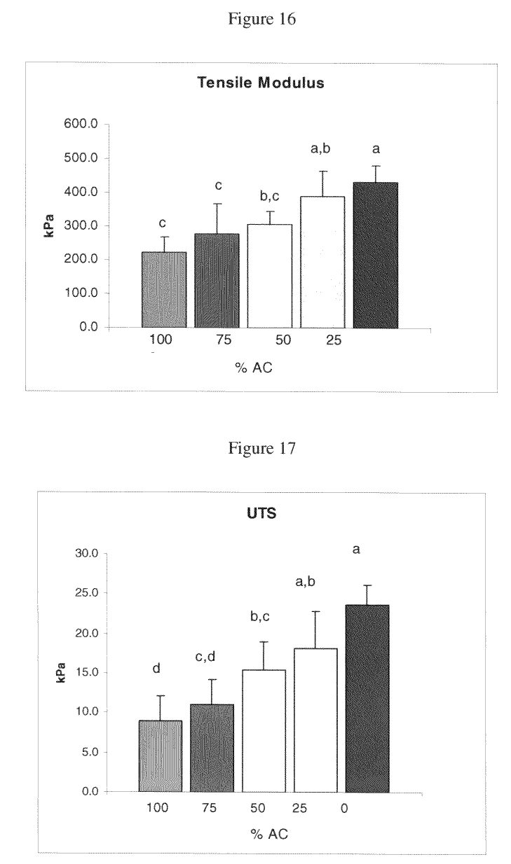

The ultimate tensile strength of the developing constructs were analyzed using know techniques. The ultimate tensile strength of the constructs also appears to increase with increasing fibrochondrocyte composition. (See FIG. 16).

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A method for forming a scaffoldless tissue engineered construct comprising:
providing a shaped hydrogel negative mold;
seeding the mold with cells;
allowing the cells to self-assemble in the mold to form a tissue engineered construct.

2. The method of claim 1, wherein two or more molds are used in a sequential fashion.

3. The method of claim 1 further comprising, exposing the cells to a pressure or a load or both.

4. The method of claim 1 wherein the hydrogel is formed from one or more of agarose, alignate alginate, and polyHEMA.

5. The method of claim 1 wherein the molds have the shape of at least a portion of a joint of a mammal, a cartilaginous tissue of a mammal, a tendon tissue of a mammal, or a ligament tissue of a mammal.

6. The method of claim 1 wherein the molds have the shape of at least a portion of a femur or a temporomandibular joint.

7. The method of claim 1 wherein the mold is in the shape of a meniscus

8. The method of claim 1 wherein the mold is in the shape of a projection of the meniscus rotated through 360 degrees.

9. The method of claim 1 wherein the cells are chosen from one or more of chondrocytes, chondro-differentiated cells, fibrochondrocytes, and fibrochondro-differentiated cells.

10. The method of claim 9 wherein the fibrochondrocytes are meniscal fibrochondrocytes.

11. The method of claim 1 wherein the cells comprise a co-culture of fibrochondrocytes and chondrocytes.

12. The method of claim 1 wherein the cells are chondro-differentiated stem cells or fibrochondro-differentiated stem cells or both.

13. The method of claim 1 wherein providing the shaped hydrogel negative mold comprises:
coating at least one surface of a culture vessel with a molten hydrogel;
inserting a shaped press into the molten hydrogel;
allowing the molten hydrogel to cool around the press; and
removing the press thereby leaving a shaped hydrogel negative mold.

14. The method of claim 1 further comprising, treating the cells with an anti-contraction agent, wherein the anti-contraction agent is staurosporine or a ROCK inhibitor or both.

15. A method for forming a scaffoldless tissue engineered construct comprising:
providing a shaped hydrogel negative mold and a shaped hydrogel positive mold;
seeding the negative mold with cells;
applying the positive mold to the negative mold; and
allowing the cells to self-assemble within the negative and positive molds to form a tissue engineered construct.

16. The method of claim 15, wherein two or more negative or two or more positive molds or both are used in a sequential fashion.

17. The method of claim 15 wherein the hydrogel is formed from one or more of agarose, alignate alginate, and polyHEMA.

18. The method of claim 15 wherein the molds have the shape of at least a portion of a joint of a mammal, a cartilaginous tissue of a mammal, a tendon tissue of a mammal, or a ligament tissue of a mammal.

19. The method of claim 15 wherein the molds have the shape of at least a portion of a femur or a temporomandibular joint.

20. The method of claim 15 further comprising, exposing the cells to a pressure or a load or both.

21. The method of claim 15 wherein the cells are chosen from one or more of chondrocytes, chondro-differentiated cells, fibrochondrocytes, and fibrochondro-differentiated cells.

22. The method of claim 15 wherein the cells comprise a co-culture of fibrochondrocytes and chondrocytes.

23. The method of claim 15 wherein the cells are chondro-differentiated stem cells or fibrochondro-differentiated stem cells or both.

24. A method for treating a subject comprising implanting in the subject a composition comprising at least one tissue engineered construct prepared by the method of claim 1 or claim 15.

25. A scaffoldless tissue engineered construct prepared by the method of claim 1 or claim 15.
